(19) United States
(12) Patent Application Publication
(10) Pub. No.: US 2009/0318307 A1
(43) Pub. Date: Dec. 24, 2009

(54) DEVICE FOR MOLECULAR DIAGNOSIS

(75) Inventor: Pablo Garcia Tello, Leuven (BE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,034

(22) PCT Filed: Jul. 17, 2007
(86) PCT No.: PCT/IB07/52842

§ 371 Date: Jan. 26, 2009

(30) Foreign Application Priority Data
Jul. 27, 2006 (EP) 06117954.5

(51) Int. Cl. C40B 30/10; G01N 27/00; G01N 21/00; G01N 33/00

(52) U.S. Cl. 506/12; 422/82.01; 422/82.05; 436/94

(57) ABSTRACT

The present invention relates to biological detection devices wherein melting curve analysis is performed an electrical sensor and a programmable heating element. The device optionally further comprises means for optically detecting nucleic acids within the device.

### FIELD OF THE INVENTION

The present invention relates to devices and methods for determining melting temperatures of double stranded nucleic acids for use as sensors of specific DNA sequences.

### BACKGROUND OF THE INVENTION

SNPs (Single Nucleotide Polymorphisms) occur when there are two or more possible nucleotides at a specific mapped location in the genome (a mismatch between base pairs in the genome of an individual). They occur in the human genome with an estimated frequency of 1 in every 1200 to 1500 base pairs (bp). Specific SNPs have been associated with diseases (i.e. gastric or peptic ulcers, cancer etc) and have even been shown to predict the response to drugs. Generally a patient's map of known SNPs is compared to a map of the same SNPs from a control group. If the pattern from the patient varies, those differences could point to a genetic factor, which could potentially be linked to the disease or the patient's susceptibility to a specific treatment.

Estimates of the number of SNPs required to create a useful map range from 100,000 (one SNP per every 30 kb of DNA) to 1 million (one SNP per every 3 kb or less). In general, the more SNPs on the map the better, and the ideal number is probably between 600,000 and 1 million. However, the number of SNPs on the map must be balanced against the cost of identifying them.

SNPs can be detected by the difference in melting temperature between completely complementary double stranded DNA (dsDNA) and dsDNA comprising a mismatch. The melting temperature is generally determined using so-called Melting Curve Analysis (MCA). MCA is performed by slowly heating double stranded DNA fragments of which one is typically labeled with a fluorescent dye. As the DNA is heated, fluorescence rapidly decreases when the melting temperature is reached and the double strand denatures [Akey et al. (2001) Biotechniques. 30, 358-362]. dsDNA wherein a mismatch occurs will denature at lower temperatures. Fluorescent melting curve analysis for genotyping SNPs on 96 or 384 well microplates is described in Bennett et al. (2003) Biotechniques 34, 1288-1294.

Nanotube sensor devices detect the binding of biological molecules, by electronic transduction of the binding through nanostructured elements, avoiding the requirement of labeling. WO2006024023 discloses nanotube sensor devices for DNA detection. It suggests the detection of SNPs, by varying the stringency upon binding (e.g. using different pH or temperatures). It is unclear however, to what extent these varying conditions affect the electrical responses of the nanotubes and whether this allows a reliable detection.

### SUMMARY OF THE INVENTION

An object of the present invention is to provide methods and apparatus for identifying differences in nucleotide sequences, such as SNPs.

The present invention discloses devices and methods wherein Melting Curve Analysis (MCA) is performed using electrical sensors, optionally combined with optical detection methods.

It is an advantage of the present invention that MCA determination via electrical measurement can be performed without the need of labeling samples with an optical label.

It is an advantage of particular embodiments of the present invention that MCA can be performed within the same device using two different detection methods (electrical measurement and optical detection).

It is a further advantage of particular embodiments of the present invention that the provided devices and methods can be used to determine accurate melting temperatures for a given double stranded (ds) nucleic acid using a combination of electrical and optical methods. This reference value can then be used in the future detection of this given nucleic acid, and allow reliable detection using only the electrical method.

It is a further advantage of the present invention that the devices and methods can be used to determine the correlation between the results obtained in the electrical method and the results obtained by MCA for different SNPs occurring in a nucleic acid. This information can be stored in a library and used as reference to allow reliable determination of SNPs using only the electrical method.

A first aspect of the invention relates to a biosensor device (100) comprising a microchamber (101), an electrical detection means (102) comprising an electric sensor placed within the microchamber, wherein said sensor is capable of detecting a change in an electric property of a double stranded nucleic acid present on its surface, and a programmable heating element (103) capable of heating the microchamber.

In one embodiment the electric sensor comprises a nanostructure such as a carbon nanotube. In another embodiment at least one single stranded nucleic acid is present on the electric sensor of the device.

In a particular embodiment the microchamber of the device comprises at least one semi-transparent or transparent portion.

In yet another one embodiment the device comprises an optical detection means (e.g. a fluoresence detector) capable of detecting a signal generated in the microchamber through the at least one semi-transparent or transparent portion of the microchamber.

In a particular embodiment, the device comprises at least two electric sensors, which are arrayed.

In yet another embodiment the device further comprises a first substrate supporting the electric sensor.

Another aspect of the invention relates to a method for performing Melting Curve Analysis, the method comprising: providing a single stranded nucleic acid probe on the surface of electric sensor in a microchamber; contacting a sample comprising a single stranded nucleic acid target with the electric sensor in the microchamber so as to allow hybridization between the single stranded nucleic acid probe and the single strand nucleic acid target to a double stranded nucleic acid; and gradually heating the microchamber and detecting the melting temperature of the double stranded nucleic acid based on a changing electrical signal on the electric sensor.

In one embodiment of the method further comprises the steps of detecting the melting temperature of the double stranded nucleic acid based on a changing optical signal and comparing the value obtained from the optical signal with the value obtained from the electrical signal.

Yet another aspect of the invention relates to methods for determining the presence of one or more nucleotide polymorphisms in a nucleic acid fragment of a gene in a sample using an electrical detection method, the method comprising the steps of determining the melting temperature of a library of nucleotide polymorphisms of the gene using an electrical detection means; for each of these nucleotide polymorphisms, simultaneously determining the melting temperature using optical detection means; correlating the values obtained with the electrical detection means with those obtained with the optical detection means so as to obtain a library of values of melting points based on electrical detection; determining the melting point for the nucleic acid fragment of the sample using an electrical detection method and comparing the value obtained with the library so as to obtain a reliable indication of the presence of the one or more polymorphisms.

Another aspect of the invention relates to a method for calibrating the devices of the present invention comprising the steps of determining the melting temperature of a double stranded nucleic acid using an electrical measurement method; verifying the melting temperature so obtained one or more times by the simultaneous determination of the melting temperature using an optical method and defining the electrical measurement values corresponding to the optically determined melting temperature of the double stranded nucleic acid.

Yet another aspect of the invention relates to a method for measuring the hybridization between a sample nucleic acid and a nucleic acid probe comprising the steps of providing an electric sensor with the single stranded nucleic acid probe; applying a sample with sample nucleic acid under conditions wherein the sample nucleic acid can hybridize with the nucleic acid probe; gradually heating the hybridized nucleic acid on the electric sensor in a controlled way and determining the melting temperature of the hybridized nucleic acid by way of the electrical sensor.

In a particular embodiment of this method, during the heating of the hybridized nucleic acid, the melting point of the hybridized nucleic acid is detected by way of optical detection. In a particular embodiment of this method, the sample nucleic acid is labeled with an optical label.

In one embodiment of this method the heating is performed at a speed of at least 1° C. per second. In a particular embodiment the detection is performed exclusively by electrical measurement using the electrical measurement values determined with the above mentioned calibration method.

Yet another aspect of the invention relates to use of the device of the present invention and the methods of the present invention for determining mismatches in a ds nucleic acid.

Yet another aspect of the invention relates to a reaction chamber of a nanosensing device comprising at least one electric sensor, a substrate supporting the electric sensor, wherein the substrate is heat conducting and wherein the microchamber comprises at least one (semi)transparent portion.

In a particular embodiment this reaction chamber further comprises a programmable heating element capable of heating the microchamber.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference Figures quoted below refer to the attached drawings.

### BRIEF DESCRIPTION OF THE FIGURES

FIG. 1 shows, in accordance with one embodiment of the present invention, a side view of a schematic configuration of a nanosensor 1: substrate; 2: nanostructure; 3: insulating layer; 4: electrodes (source and drain); 5: gate electrode.

FIG. 2 shows, in accordance with one embodiment of the present invention, a top view of schematic configuration of an arrayed nanosensor with built in heating elements (6), (3) is an optional material that covers the heating element and provides isolation; 2: nanostructure, 4: electrodes,

FIG. 3 shows, in accordance with one embodiment of the invention, a top view of schematic configuration of an arrayed nanosensor with built in heating elements (6) and transparent or semi-transparent portion (7); 2: nanostructure, 3: insulating layer, 4: electrodes.

FIG. 4 shows, in accordance with one embodiment of the invention, a theoretical example of a MCA. A: complementary ds nucleic acid, B: ds nucleic acid with one mismatch: C theoretical melting curves of the ds nucleic acids in A and B.

FIG. 5 shows the elements of a biosensor device 100 in accordance with one embodiment of the invention wherein 101: microchamber, 102: electrical detection means, 103: heating element, 104: optical detection means, 105: providing means, 106: sources, 107: control and analysis circuitry, 108: input/output means.

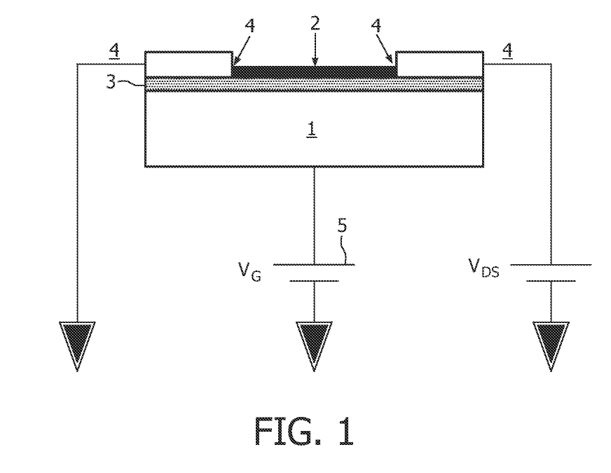

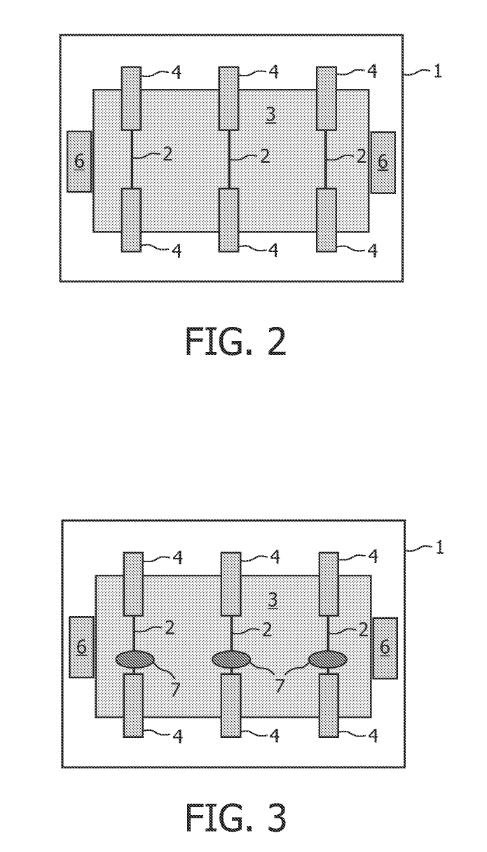

In the different Figures, the same reference signs refer to the same or analogous elements.

### DETAILED DESCRIPTION OF AN EMBODIMENT

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. These definitions should not be construed to have a scope less than understood by a person of ordinary skill in the art.

The term “polymorphism”, as used herein refers generally to the ability of an organism or gene to occur in two or more different forms. In the present invention, “polymorphism” refers in particular to two or more different forms of the same gene.

The term “Single Nucleotide Polymorphism” or “SNP” as used herein, refers to a polymorphism that results from a difference in a single nucleotide.

The term “allele” refers generally to any of one or more alternative forms of a given gene or nucleic acid segment; both or all alleles of a given gene are concerned with the same trait or characteristic, but the product or function coded for by a particular allele differs, qualitatively and/or quantitatively, from that coded for by other alleles of that gene. Three or more alleles of a given gene constitute an allelomorphic series. In a diploid cell or organism the members of an allelic pair (i. e., the two alleles of a given gene) occupy corresponding positions (loci) on a pair of homologous chromosomes ; if these alleles are genetically identical the cell or organism is said to be homozygous. If the alleles are genetically different, the cell or organism is said to be heterozygous with respect to the particular gene. A wild type allele is one which codes for particular phenotypic characteristic found in the wild type strain of a given organism.

In a “melting profile”, a parameter (X) affected by the transition of a double strand nucleic acid to a single strand nucleic acid is plotted vs. temperature (T). Classically, this parameter is an optical signal (such as fluorescence (F)) ensured by a label which is only present in or characteristic of the double stranded nucleic acid (such as intercalating agents and labels bound to one of the strands, as described herein).

The term “melting temperature” or “Tm” refers to the temperature at which 50% of a double strand nucleic acid has denatured into single strand nucleic acid. The Tm corresponds to the midpoint between the minimum signal and maximum signal in a melting profile and to the apex of a peak in the negative first derivative (−d(X)/dT) of a melting profile of a nucleic acid.

The term “nucleic acid”, as used herein includes deoxyribonucleic acids, oxyribonucleic acids, oligonucleotides, polynucleotides, ribonucleic acid, messenger ribonucleic acid, transfer ribonucleic acid, and peptide nucleic acid and other synthetic counterparts.

The term “single-stranded (ss) nucleic acid” refers to a single strand of a nucleic acid as defined above.

The term “double-stranded (ds) nucleic acid” as used herein a double strand of a nucleic acid described above, including hybrid ds nucleic acids such as RNA/DNA. It includes hybrids whereby only a part (at least ten nucleotides) of one or both strands are complementary and form a double strand.

The term “nucleic acid probe” refers to nucleic acid which is present on the surface of a sensor, such as a nanotube, prior to hybridization and detection. Typically the nucleic acid probe is selected so as to allow hybridization with a particular target nucleic acid believed to be present in a sample.

The term “sample nucleic acid” as used herein refers to a nucleic acid which is present in a sample.

The term “electric sensor” as used herein refers to a sensor capable of detecting a change in an electric property of a double stranded nucleic acid present on its surface thereto. Examples of such electric properties include resistance, impedance, conductivity.

The term “nanostructure” as used herein refers to an object that has at least one dimension smaller than 100 nm and comprises at least one sheet of crystalline material with graphite-like chemical bonds.

The term “nanosensor” as used herein is a detection device whereby detection occurs through a sensor which is a nanostructure or a network of nanostructures.

The term “nanotube” as used herein refers to a single tubular-shaped element, multiple tubes or a network of interconnected tubes of nanostructures diameter of between 1 to 2 nm.

When referring to “Electric measurement” herein it is intended to refer to the measurement of one or more electrical properties such as resistance, impedance, transconductance, capacitance, etc.

The present invention relates to methods and tools for the detection of biomolecules, more particularly for the detection of nucleic acids, based on the strength of hybridization with a second nucleic acid. More particularly, the present invention provides methods and tools for the reliable determination of the melting temperature (Tm) of a ds nucleic acid.

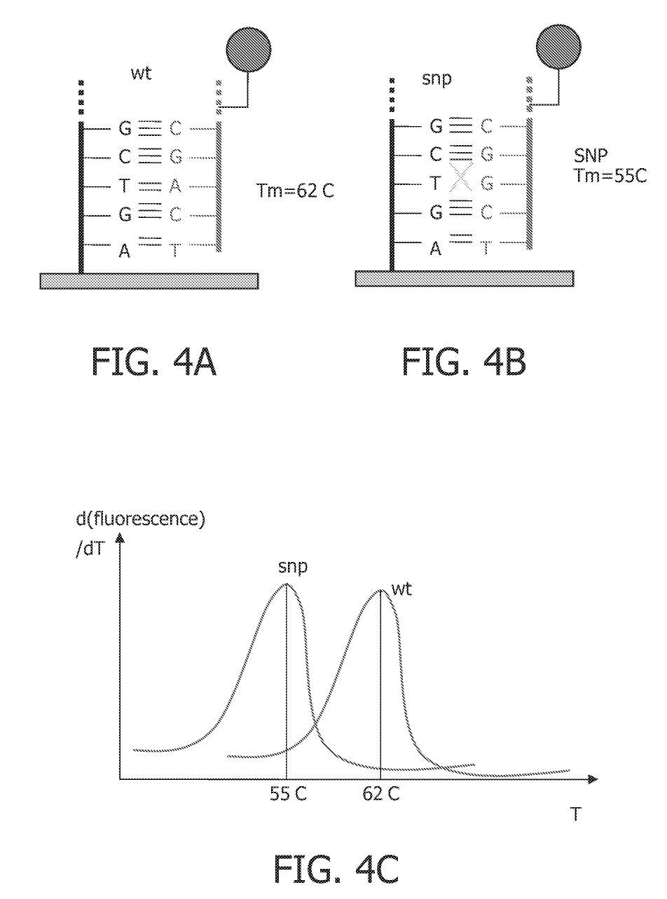

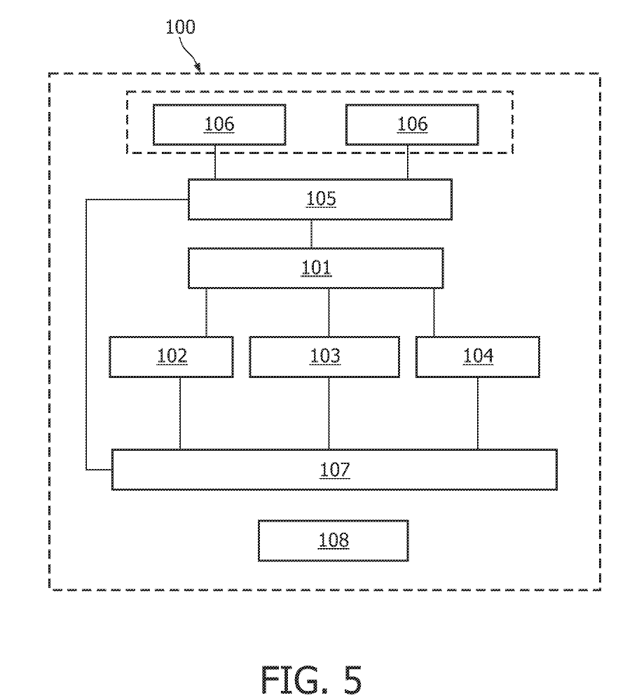

The value of the melting temperature is dependent on a number of factors, including the GC content. More specifically, the proportion of GC pairs in the nucleic acid is relevant to the Tm. GC pairs, having three hydrogen bonds, are more stable than AT pairs which have only two hydrogen bonds. Thus, it is possible to distinguish DNA fragments that differ with respect to their GC/AT ratio by melting curve analysis (MCA). The concept of melting curve analysis is described in detail in Akey (cited above). Melting curve analysis is performed by measuring a parameter indicative of the decrease of double stranded nucleic acid (inherent to the double stranded nucleic acid or as a result of the presence of a label) and/or the increase of single stranded nucleic acid (generally referred to as ‘X’), as a function of temperature. This parameter is affected when the melting temperature (Tm) of the double stranded nucleic acid is reached, due to denaturation of the double stranded nucleic acid. By taking the negative first derivative of this parameter (−dX/dT), the melting temperature of a ds nucleic acid can be easily visualized and compared, simplifying the discrimination between fully complementary ds nucleic acids and ds nucleic acids with one or more mismatches. Typically, where the parameter is the binding of an intercalating agent (see below) the parameter is fluorescence and the negative first derivative of the fluorescence is used (−dF/dT) (See FIG. 4)

The invention relates to detection devices based on electrical sensors wherein heating elements are provided to gradually heat (and optionally cool) the sample present on the electrical sensor in a programmable and reliable way. For the determination of the melting temperature of a double stranded nucleic acid, a controllable temperature gradient is applied (e.g. between 0.1° C./s to 1° C./s) which is typically within a range of about 20 to 100° C., more particularly between 20 to 80° C. Heating to higher temperatures up to 100° C. can be used to denature nucleic acids, to destroy the activity of proteins present in a sample or to denature enzymes such as used in the amplification or labelling of nucleic acids. Cooling to about 4° C. can be used to anneal/hybridize nucleic acids, or to store the sample prior or after analysis.

The controllable heating elements of the devices of the present invention can include temperature sensors. Data from the temperature sensors an be provided to a data analyzer for combination with the data of the electrical and/or optical detection means described herein. Optionally a temperature measuring means for detecting the temperature within the microchamber is provided as separate unit.

According to one embodiment, the device comprises one or more heating elements which allow the controlled heating of the surface of the one or more sensors and/or a microchamber comprising the one or more sensors. Heating elements for heating and cooling of containers or chambers comprising nucleic acid samples in a controllable and accurate manner for use in the device and methods of the present invention are known in the art. Such heating elements include electric heaters, thermoelectric heaters and coolers (Peltier devices), resistive heaters, capacitively coupled RF heaters, heat sinks, fluidic circuit heaters, heatpipes, chemical heaters, and other types. In certain embodiments, heating is performed using an off-board heating mechanism. In other embodiments the heating mechanism does not come into physical contact with the microfluidic device. For example, electromagnetic radiation may be used to heat the interior of the microchamber, such as the radiation which is within the microwave spectrum or within the infrared spectrum. Alternatively, an external heating mechanism is used in contact with the microchamber, such as (ultra)sonic heaters used to induce heating of a fluid.

The one or more heating elements are placed so as to ensure appropriate heating of one or more of the sensors and/or areas surrounding the sensors and/or the microchamber. Typically the one or more heating elements are provided within a substrate which provides the necessary isolation. For example heating elements, e.g. resistors, are micromachined directly in the substrate by using well-known semiconductor fabrication techniques as lithography and etching. Also the same techniques are used optionally to provide the suitable isolation to confine the heat within the desired sensor area. According to one embodiment the heating elements are surrounded by deposited layers of materials that will act as heat sink elements. In a particular embodiment the heating element is a resistor with zig-zag line conformation which is embedded in the substrate and that runs underneath the nanotubes and is properly isolated to avoid electronic disturbances.

The present invention may also optionally include cooling elements in the microchamber. As an example, an active cooling element may be a Peltier element. Any form of microcooling device can be used. For example one type of cooling devices are micro-electro-mechanical refrigeration systems. One example of such a system may be a refrigeration system based on a magnetic refrigeration cycle whereby a micro-electro-mechanical switch, a micro relay, a reed switch or a gate switch is used for switching between an absorption phase and a heat rejection phase of such a cycle. Such devices are described in more detail in e.g. U.S. Pat. No. 6,588,215 B1 from International Business Machines Corporation. Another example of such a system may be a thermoacoustic refrigerator based on providing a temperature difference across a stack using a piezoelectric driver. Thereby a high frequency sound is generated which, by interaction with one or more parts of the stack creates a temperature gradient, thus allowing cooling, as e.g. described in more detail in U.S. Pat. No. 6,804,967 B2 by University of Utah. Still another example of such a system may be a micro-electro-mechanical system whereby expansion of gas is controlled using a micro-electro-mechanical valve, as described in more detail in U.S. Pat. No. 6,804,967 by Technology Applications, Inc. It is an advantage of several of these cooling means that they can be applied using micro-electro-mechanical technology, lithography or thin film deposition techniques such that they can be integrated in the microchamber and their size is compact.

The present invention relates to detection devices based on electrical sensors. Accordingly, such devices typically include an electrical detection means (102) which is capable of registering the signal generated by an electric sensor. According to one embodiment of the present invention, devices are provided wherein one or more electric sensors are provided. Where independent detection on multiple electric sensors (e.g. arrays of electrical sensors) is desired, it is envisaged that independent heating elements can be placed in the vicinity of each of the electric sensors can deliver heat at a an appropriate and optionally different rate.

According to one embodiment, the sensors are electrical detection means further comprising a source electrode, a drain electrode, and optionally a gate electrode. For example, in the presence of a gate electrode, the electrical sensor connects the source and the drain to form a field-effect transistor.

According to one embodiment of the invention, the electrical sensor is a nanostructure, i.e. a structure which has at least one dimension smaller than 100 nm. According to one embodiment, the nanostructure comprises at least one sheet of crystalline material with graphite-like chemical bonds, such as for example single and/or multiwalled carbon nanotubes double-walled nanotubes, multi-walled nanotubes, or “onions” and/or interconnecting networks comprising such nanotubes which interact with polynucleotides so as to act as sensing elements. Typically the nanostructure is arranged on a substrate (a first substrate), which can be a sidewall of a microchamber.

Nanotubes for use in the context of the present invention may be single-walled carbon nanotubes (SWNT), having a diameter of between 1 to 2 nm. The nanotubes can comprise a single tube, multiple tubes or a network of interconnected tubes. According to one embodiment, the nanotubes are multi-walled nanotubes (MWNT). In one embodiment, the multiple nanotubes are oriented parallel to each other on the substrate. Alternatively, the multitude of nanotubes are oriented randomly. The number of nanotubes in an area of substrate is referred to as the density. Where the substrate comprises many nanotubes oriented randomly, the density should be sufficiently high to ensure that electric current passes through the network from one side of the defined area to the other side, such as via nanotube-to-nanotube contact points. While nanotubes are mostly made of carbon, other materials e.g., silicon nanowires and inorganic nanorods boron nitride, molybdenum disulfide and tungsten disulfide may also be used. The nanotubes may be semiconducting depending on the chirality of the nanotube. Methods of growing nanotube networks are known to the skilled person and include methods such as chemical vapor deposition (CVD) with traditional lithography, solvent suspension deposition, vacuum deposition, and the like (WO2004040671 and Hu et al. (2004) Nano Lett. 4, 2513-2517). In areas outside the area between the opposing electrodes, excess nanotubes can be removed from the substrate using suitable methods, such as plasma etching.

According to yet another embodiment, the electrical sensor comprises one or more electrodes of metal or a metal alloy. For example, the electrodes may be of Ti, Pd, Au.

In order for the sensor to transfer the change in electrical property of the nucleic acid present on its surface to an electrical signal which can be detected, the sensor is optionally connected to an electrical circuit through contacts. A contact includes a conducting element which ensures the electrical communication with the sensor. Where the sensor is a nanostructure, contacts may be disposed directly on the surface of the first substrate which supports the nanostructure, or alternatively may by disposed over the nanostructure, e.g. over a nanotube network. Electric current flowing in the nanotube network may be measured by employing at least two contacts that are placed within the defined area of the nanotube network, such that each contact is in electrical communication with the network. An additional contact which is not in electrical communication with the sensor may be provided as gate electrode, such that there is an electrical capacitance between the electrode and the sensor. Typically, this gate electrode is placed below a substrate supporting the sensor. Examples of such nanodevices are provided for example in US2004132070. Different electrical properties of the sensor including resistance, impedance and transductance can be measured under the influence of a selected or variable gate voltage. Voltage can also be applied to one or more contacts to induce an electrical field in the sensor relative to a gate electrode, and the capacitance of the network can be measured

The devices of the present invention comprise a first substrate, which mechanically supports the electric sensor (either by suspension or by direct support).

Where the electrical sensor is a nanostructure, this sensor is typically fabricated on the surface of a substrate, which is of an electrically insulating material, for example silica based. The area of surface of the substrate covered by each of the sensors is typically no more than about 1 cm. Most particularly, the area of the substrate surface covered by each of the sensors is between about 1 mm² and about 0.01 mm². In a particularly preferred embodiment, each sensor covers an area of the substrate surface from about 100 μm² to about 2,500 μm². In a particular embodiment, the sensors are placed as an array (see below) which is contained within an area of about 3 cm² or less on the surface of the substrate.

Alternatively, the substrate is of an electrically conducting material, for example silicon or metal, provided that there is an electrically insulating layer between the conducting substrate and the electrical sensor.

Suitable substrates can be of silicon oxide, silicon nitride, aluminum oxide, polyimide, and polycarbonate. In a number of examples described herein, the substrate includes one or more layers, films or coatings comprising such materials as silicon oxide, SIO₂, Si₃N₄, and the like, upon the surface of a silicon wafer or chip. According to one embodiment, the first substrate is of a transparent material, such as SIO₂ or a material which can be made transparent.

As indicated above, where the electrical sensor is placed in a microchamber, the first substrate is either used to form one or more walls of the microchamber or is provided as a coating of one or more of the microchamber walls.

According to one embodiment of the invention the heating elements of the device are provided below the surface or on top of the surface of the first substrate, so as to facilitate the heating of the electric sensor, the area surrounding the electric sensors, and/or the liquid in the microchamber of the device.

According to a particular embodiment, the devices of the present invention further comprise a layer of insulating material, which is deposited over the contacts of the electrical sensor. Various suitable polymers and resins are known in the art, including epoxy coatings. The insulating layer may be of similar material as the insulating material of the first substrate. It can be selectively applied on certain areas of the first substrate comprising the contacts, or can be applied over the entire substrate and removed from operative areas of the sensor such as between the contacts. The insulating layer provides for electrical insulation, preventing short-circuiting of the sensor when in contact with a conductive fluid such as a buffer used for application of a sample, or otherwise protecting the sensor from exposure to the environment. The insulating layer may also be helpful in controlling the deposition of other materials, including but not limited to nanotubes and DNA molecules. Any number of insulating layers may be used.

The devices of the present invention an comprise at least one reaction chamber or microchamber (101) comprising the electric sensor used in the electrical detection means. Typically the size of a microchamber is between 1-100 μl. According to one embodiment, one microchamber comprises more than one electric sensor, allowing simultaneous measurements with different nucleic acid probes. According to a particular embodiment, multiple sensors are arrayed within a microchamber. An array can be a one-dimensional or two-dimensional arrangement in a predefined way of a plurality of sensors. The sensors can be arrayed in one single reaction chamber. Alternatively, the sensors are spread over different reaction chambers. Typically, an array comprises at least ten sensors. In a particular embodiment, the array comprises at least about 50 sensors, more particularly about 100 sensors, most particularly more than 10³, 10⁴ or 10⁵ sensors.

According to one embodiment of the invention at least one wall of the microchamber is formed by or coated with a first substrate, having the properties described above. According to a particular embodiment, the microchamber is formed by an opening which has been etched in the first substrate. The microchamber is optionally further partially coated with one or more insulating layers as described above.

Additionally or alternatively, at least one part of the microchamber walls comprises a transparent portion to enable optical detection within the microchamber, more particularly on the electrical sensor. Typically this is ensured by a structure which is of a material such as SiO₂, plastic or PVC. As indicated above, this portion can be an integral part of the first substrate. Alternatively, the transparent or semi-transparent portions can be provided in any of the walls of the reaction chamber, provided that it allows optical detection of the area of the sensor, wherein the melting curve analysis is performed

According to one embodiment, the microchamber is an integral part of the devices of the present invention. Alternatively, the microchamber is provided as a separate, optionally disposable cassette or cartridge, for use in a device comprising the required connections for detecting the electrical signal and optionally the optical signal on the surface of the electric sensor. According to one embodiment, the microchamber comprises at least one electric sensor, and a substrate mechanically supporting the electric sensor, wherein at least the substrate is heat conducting. More particularly, the microchamber allows for heating of the microchamber by heating means placed outside the microchamber, e.g. in a detection device. According to one embodiment the heating device is placed within the microchamber wall, or the microchamber wall comprises elements capable of generating heat within the microchamber in a controllable way. Optionally, the heating elements are controlled when placed e.g. in a device with appropriate contacts for the elements, by control means of a detection device.

The microchamber of the devices of the present invention typically comprise one or more in and outlets, for the introducing and/or removing of samples, buffers, etc.

Typically, the microchamber of the device is integrated in a microfluidic system which allows the delivery and flow of minute amounts of fluids to/from the microchamber, and more particularly to/from the electric sensor(s). Accordingly, the devices of the present invention further comprise a providing means (105) for providing sample, buffer, reagents and/or additives from one or more sources (106) to the reaction chamber and/or the electrical sensor. The means may include gravimetric feeds of the sample and may also include an arrangement of pipes/conduits, mixers and valves, e.g. selectable and controllable valves, to allow the provision of fluids from different sources to the microchamber and from the chamber to one or more collectors and/or a waste. Microfluidic devices based on capillaries typically have cross-sections of 10-100 μm. Both simple two-dimensional and more complex three dimensional systems of pumps, valves, and channel systems are envisaged.

The microfluidic systems or devices may be fabricated in silicon, glass and polymers (Microsystem Technology: A Powerful Tool for Biomolecular Studies, Kohler, J. M. et Al. Edsl, Birkhauser Verlag, Boston (1999)). Polymer microfluidics such as Poly(dimethylsiloxane) (PDMS) are particularly attractive in prototyping new systems because fabrication of systems of channels in PDMS is straightforward and can be cast against a suitable mold with sub-0.1-micron fidelity (McDonald et al (2002) Act. Chem. Res. 35, 491-499).

According to one embodiment, mechanical micropumps and valves within the device move fluids within microfabricated devices (such as described e.g. in U.S. Pat. No. 5,271,724 and U.S. Pat. No. 5,277,556).

Alternative methods have been described and are known to the skilled person for the transport and direction of fluids, e.g., samples, analytes, buffers and reagents within these microfluidic systems or devices. Such methods include the application of external pressure to move fluids within the device (U.S. Pat. No. 5,304,487), the use of acoustic energy to move fluid samples within devices by the effects of acoustic streaming (such as described e.g. in WO9405414). Yet another method uses electric fields to move fluid materials through the channels of the microfluidic systems (such as described e.g. by Harrison et al. (1992) Anal. Chem. 64, 1926-1932 and in U.S. Pat. No. 5,126,022)

The integration of a microfluidic system into a biological sensor working with microliter to nanoliter volumes is beneficial because the use of volumes smaller than 10 μl generates significant problems with evaporation, dispensing times, protein inactivation, and assay adaptation. Miniaturization of assays to volumes smaller than 1 μl increases the surface to volume ratio substantially. Furthermore, solutions of submicroliter volumes evaporate rapidly, within seconds to a few minutes, when in contact with air.

Typically, the devices of the present invention further comprise control circuitry which ensures the control of the providing means.

According to a particular embodiment, the devices of the invention also allows MCA using optical detection. Accordingly, the devices according to this aspect of the invention comprise a microchamber of which at least one wall has a transparent or semi-transparent portion (or window) which allows the detection of an optical signal within the microchamber, more particularly in the region of the sensor. Alternatively, the device is conceived so that introduction of a microchamber cartridge comprising at least one wall which has a transparent or semi-transparent portion (or window) ensures that optical detection of a signal within the microchamber is possible. Where optical detection is required the devices of the present invention comprise an optical detection means (104), such as a fluorescence detector. The nature of the detection means is determined by the nature of the dye or label used. Suitable optical detection means are well known to the skilled person.

According to the present invention, the optical detection means allows for the determination of an MCA in parallel with or as the calibration of the electrical detection of an MCA for a nucleic acid. Additionally it is envisaged that the optical detection means can be used for the identification and/or quantification of nucleic acids within the sample

Both the electrical and optical detection means may be under the control of the control and analysis circuitry (107). Signals representative of the detections may be supplied to the control and analysis circuitry which can be adapted to carry out any of the MCA of the present invention described above.

The control and analysis circuitry conventionally includes a connection with the detection means and providing means to evaluate the detection signal corresponding to the Tm of the target. The system may further provide statistical processing of the obtained detection results, e.g. to correlate two different measurements of each detection system or between different detection systems.

The control and analysis circuitry may also include means for determining that the sample nucleic acid has been received by the nucleic acid probe on the sensor, and that the amount of sample nucleic acid is sufficient for testing. The control and analysis circuitry may comprise a processing means, such as e.g. a microprocessor, and/or a memory component for storing the obtained and/or processed evaluation information.

Furthermore the devices of the present invention may further comprise typical input/output (108) means. The control and analysis circuitry may be controlled using appropriate software or dedicated hardware processing means for executing the evaluation steps. The control and analysis circuitry may thus be implemented in any suitable manner, e.g. dedicated hardware or a suitably programmed computer, microcontroller or embedded processor such as a microprocessor, programmable gate array such as a PAL, PLA or FPGA, or similar. The control and analysis circuitry typically will store and display the results of the analysis on any suitable display means such as a visual display unit, plotter, printer, etc. or may alternatively provide the data to a separate device. The control and analysis circuitry may also have a connection to a local area or wide area network for transmission of the results to a remote location.

Control and analysis circuitry may be at least partly provided as a separate cartridge comprising a microchamber or may optionally be external to the cartridge and may be provided optionally to control the operation of the providing means. The control and analysis circuitry may be connected to the providing means by suitable contacts on the surface of the cartridge, e.g. terminals.

According to the present invention, detection by an electric sensor is based on the changing property of nucleic acids present on the surface of the sensor upon transition from a ds nucleic acid to a ss nucleic acid. Different electrical properties are envisaged to be detected using the electric sensors of the invention including electrical resistance, electrical conductance, current, voltage, capacitance, transistor on current, transistor off current, or transistor threshold voltage. They may be measured under the influence of a selected or variable gate voltage. Conveniently, the source (and/or drain) and gate electrodes of a transistor based on a channel formed by the electric sensor (such as e.g., a nanotube network) may be employed using suitable circuitry to measure the capacitance of the channel relative to the gate, as an alternative or additional sensor signal to measurements of one or more channel transconductance properties. In another particular embodiment, the gate electrode is a conducting element in contact with a conducting liquid, this liquid being in contact with the sensor. An example hereof is described in Bradley et al. (2003) Phys. Rev. Lett. 91, 218301.

In a particular embodiment, the biosensor device includes a transistor. A transistor has a maximum conductance, which is the greatest conductance measured with the gate voltage in a range, and a minimum conductance, which is the least conductance measured with the gate voltage in a range. A transistor has an on-off ratio, which is the ratio between the maximum conductance and the minimum conductance.

The present invention provides methods and devices for detecting electrical properties of nucleic acids using electric sensors. According to one embodiment, selective detection of a nucleic acid is ensured by providing a nucleic acid probe on the surface of the electric sensor. Nucleic acids may be attached directly to the electric sensor, or may be present on the surface of the first substrate supporting the electric sensor, in the vicinity of the electric sensor. Additionally or alternatively, nucleic acid molecules are provided on a material covering the electric sensors, provided the electric properties of the nucleic acids can be transferred to the electric sensor. The nucleic acid should, when electric measurements are performed be sufficiently close to the electric sensor so that a change in one or more electrical properties (e.g. as a result of a melting of the ds nucleic acid to a single nucleic acid) can be detected by the sensor.

According to one embodiment, the nucleic acid probe is provided on the sensor area only and electrical and optical detection are performed on the sensor area. Alternatively, it can be envisaged that nucleic acid prove is provided on regions of the first substrate outside the sensor area. While this nucleic acid probe will not contribute to the electrical signal, it may be used or contribute to the optical detection.

According to one embodiment, nucleic acid, more particularly single stranded nucleic acid, and most particularly a nucleic acid probe is provided on the surface of the electric sensor. Ss nucleic acid can bind to nanostructures such as nanotubes without activation of the nanostructures. Methods for providing a single stranded nucleic acid probe on the electric sensor can comprise the steps of placing a drop of nucleic acid probe solution on sensor area; evaporating the solution by drying; rinsing the electric sensor with water and drying of the sensor surface, e.g., with nitrogen. Excess probe ss nucleic acid may be removed by rinsing and blowing. More aggressive methods, e.g., etching, are used if excess probe nucleic acid is bound to undesired areas, e.g. areas of the first substrate outside of the sensor area. Alternatively, excess probe nucleic acid may be left in place if it does not disrupt sensor operation, or if it is desired for supplementary optical detection.

According to a particular embodiment, one or more nucleic acids such as a nucleic acid probe is immobilized on the surface of an electric sensor. Several methods for the immobilization of nucleic acids have been described for the preparation of DNA sensors, including chemical adsorption (Hashimoto, K.; et al., Anal. Chim. Acta 1994, 286: 219; Zhao, Y. D.; et al. J. Electroanal. Chem. 1997, 431: 203) and covalent bonding (Liu, et al. Anal. Biochem. 2000, 283, 56; Xu, C. et al. Anal. Chem. 2001, 369, 428; Steel, A. B. et al. Bioconjug. Chem. 1999, 10: 419). New immobilization techniques such as avidin-biotin system (Sun, X. Y.; et al., Talata 1998, 47: 487) and chitosan-modified electrode (Berney, H.; et al. Sensors and Actuators B 2000, 68:100) and covalent binding through molecular self-assembly (Hagenstrom, H. et al. Langmuir 2001 17, 839) have been shown to be both simple and versatile. The self-assembled monolayer (SAM) modified electrode establishes a stable, highly dense and orientable ssDNA modified monolayer. Methods for providing a plurality of different probe nucleic acids on a substrate in an arrayed manner are known from microarray technology (e.g. adapted inkjet printing devices).

According to one embodiment, after providing the nucleic acid on the sensor surface, the surface of the sensor is treated with a blocking agent, such as e.g. Triton X-100™, to prevent binding of non-relevant nucleic acids or other components to the sensor.

The present invention provides methods and tools for the detection of nucleic acids in a sample.

According to a particular embodiment the sample is a biological sample comprising DNA, more particularly the sample comprises genomic DNA from eukaryotic organisms. Most particularly the samples of interest for MCA detection are samples of genomic DNA obtained from organisms of the same species believed to carry one or more polymorphisms of one or more nucleotides in one or more genes.

According to one embodiment, the methods and devices of the present invention are for use in the detection of nucleotide polymorphisms. Nucleotide polymorphisms in a gene or gene fragment can be detected by the difference in melting temperature for a DNA sequence comprising the mismatch of between 5 and 100 nucleotides, between completely complementary double stranded DNA (dsDNA) and dsDNA comprising a mismatch. The melting temperature is generally determined using so-called Melting Curve Analysis (MCA). MCA is performed by slowly heating double stranded nucleic acid fragments obtained by hybridizing a probe corresponding to a region of the sample nucleic acid comprising the mismatch and a sample nucleic acid. The difference in Tm between a ds nucleic acid control (control nucleic acid not comprising the mismatch hybridized with nucleic acid probe) and the ds nucleic acid formed by hybridizing the sample nucleic acid with the nucleic acid probe is indicative of the presence of one or more nucleotide polymorphisms.

Prior to the analysis of a sample the nucleic acids in the sample can be amplified by various methods. Apart from PCR, other amplification methods available in the art may also be utilized, including, but not limited to target polynucleotide amplification methods such as self-sustained sequence replication (3SR) and strand-displacement amplification (SDA), methods based on amplification of a signal attached to the target nucleic acid, such as “branced chain” nucleic acid amplification, methods based on amplification of probe nucleic acid, such as ligase chain reaction (LCR) and QB replicase amplification amplification (QBR), and various other methods such as ligation activated transcription (LAT), nucleic acid sequence based amplification (NASBA) repair chain reaction (RCR) and cycling probe reaction (CPR).

According to one embodiment, the nucleic acids for which MCA is performed in the methods and devices of the present invention are in the range of 50-150 base pairs in length. It is known that the size of nucleic acids is an important factor in MCA. More particularly, there is a greater difference in the Tm between smaller nucleic acid fragments, differing in one or more SNPs, than there is for larger nucleic acid fragments. Theoretical and empirical studies of nucleic acid denaturation have shown that, as the size of nucleic acid fragments increases, the difference in Tm, generated by one or more SNPs, decreases.

According to a further particular embodiment, a destabilizing agent is added to the sample or to the hybridized nucleic acid probe/sample nucleic acid. A destabilizing agent destabilizes ds nucleic acids, thereby lowering the Tm. The addition of a destabilizing agent can ensure that the Tm of a nucleic acid, which would normally be above 100° C., falls within the temperature range reached during the experiment (i. e., Tm<100° C.). Different destabilizing agents have different influences on the shape of a melting curve of a given nucleic acid. For example, the addition of urea as a destabilizing agent results in both a change in the Tm as well as a broadening of the peaks obtained in the negative first derivative of the detection parameter (e.g. for fluorescence −dF/dT). It also decreases the fluorescence of a labeled sample. The use of DMSO and formamide results in more sharply defined peaks compared to urea. Other suitable destabilizing agents include compounds which denature ds nucleic acid by altering salt concentrations of the buffer wherein the ds nucleic acid is present. The need for the addition of a destabilizing agent can be determined experimentally or theoretically. Theoretical prediction of the Tm of a nucleic acid has been the subject of numerous studies. Melting temperatures are often calculated to determine the expected temperature range wherein the MCA is performed. Melting temperatures are predicted using salt adjusted formulas (Rychlik and Rhoads (1989) Nucl. Acids Res. 17, 8543-8551; Sambrook, J. et al. (1989) Molecular Cloning: A Laboratory Manual, Cold Spring Harbor Laboratory Press, Cold Spring Harbor, N.Y.) or nearest neighbor algorithms (Breslauer et al. (1986) Proc Natl Acad Sci. 83, 3746-3750.

The methods of the present invention are based on the detection of changing electrical properties of a nucleic acid upon transition from a double stranded to a single stranded nucleic acid. Most particularly this detection is performed during the heating of the double stranded nucleic acid, whereby the temperature at which transition occurs, i.e. the melting temperature, of the double stranded nucleic acid is determined. By use of a nucleic acid probe, single stranded nucleic acid within a sample can be captured and the electrical properties of the formed double stranded nucleic acid is indicative of the nature of the bound single strand.

According to this aspect of the invention, the detection of the melting temperature of one or more nucleic acid samples is performed, which allows the identification of the sample nucleic acid without the need for labeling.

According to another aspect of the invention, devices and methods are provided wherein the electrical detection method of the melting point of a nucleic acid sample is combined with an optical detection. According to this aspect of the invention, a labeled sample DNA is introduced into the reaction chamber, whereafter conditions are applied for (denaturation and) hybridization with the nucleic acid probe. After the hybridization of the sample nucleic acid with the nucleic acid probe, an increasing temperature gradient is applied. During the gradually increasing heating conditions, the electrical and optical properties of the hybridized ds nucleic acid are detected. More particularly, the change in electrical and optical properties upon transition of a ds nucleic acid to an ss nucleic acid is monitored, so as to determine the melting point of the ds nucleic acid.

Where the methods and devices of the present invention include optical detection, this is based on a difference in optical properties of ds and ss nucleic acids. Classically, melting point analysis has been performed by detection of the nucleic acid at 260 nm, as the absorbance of ss and ds nucleic acid at this wavelength can be distinguished. However, in order to allow more sensitive and accurate melting point determination, suitable dyes or labels can be used, which generate a differential signal for ds and ss nucleic acid.

According to one embodiment, use is made of a dye which non specifically binds to either ss nucleic acid or ds nucleic acid. Suitable dyes include (but are not limited to) a dsDNA specific dye such as ethidium bromide, SYBR Green I or SYBR Green II (Molecular Probes, Eugene, Oreg.), or a ssDNA specific dye. According to a particular embodiment, the dye is a fluorescent dye, which is detected using a fluorescence detection means. Suitable dyes can be added to the sample or can be added to the hybridized ds DNA on the sensor surface after hybridization.

Alternatively, use is made of a label, which is e.g. bound to the sample nucleic acid, whereby hybridization with the nucleic acid probe present on the sensor will generate a signal on the sensor surface. The sample nucleic acid can be modified with a label prior, during (with labeled primers) or after an amplification step. In certain embodiments two different labels can be present. For example sample and probe nucleic acid can contain a label that emits light at a different wavelength. According to one embodiment sample and probe nucleic acid are provided with labels, which, when in proximity of each other, quench or enhance a signal generated by the individual labels. The nature of the label(s) used, will determine the nature of the melting temperature analysis, i.e. this can be performed by measuring a loss in optical signal or a gain in optical signal when ds nucleic acid denatures. A non limiting list of suitable labels includes fluorescein dyes, such as 5- (and 6-) carboxy-4′,5′-dichloro-2′,7′-dimethoxy fluorescein, 5-carboxy-2′,4′,5′,7′-tetrachlorofluorescein and 5-carboxyfluorescein, rhodamine dyes such as 5- (and 6-) carboxy rhodamine, 6-carboxytetramethyl rhodamine and 6-carboxyrhodamine X, phthalocyanines such as methyl, nitrosyl, sulphonyl and amino phthalocyanines, azo dyes, azomethines, cyanines and xanthines such as the methyl, nitro, sulphano and amino derivatives, and succinylfluoresceins. Other suitable labels are fluorophores from the group of cyanine dimers and monomers, such as TOTO, YOYO, TO-PRO, Cy3, Cy5, Cy5.5, Cy7 etc., or dyes such as LCRed 705 may be used as the fluorescent dye.

According to a particular embodiment, the determination of the melting point is further facilitated using Melting Point Markers (MPMs). MPMs are specific nucleic acid fragments to be included in melting curve analysis. These fragments are small pieces of nucleic acid (cloned, synthetic, or co-amplified with the test locus) that will also melt during the heating phase acting as internal standards in a fashion analogous to molecular weight markers in electrophoresis. Using these MPMs one can have both internal verification of the melting run and an internal reference against which to compare the tested fragments.

The devices and methods of the present invention provide for performing a Melting Curve Analysis simultaneously and under the same conditions based on electrical measurements and optical measurements. This provides several advantages. For instance, it makes it possible to correct for possible deviation in the electrical measurement due to fluctuations in pH or ionic concentration. Accordingly the present invention provides for methods wherein MCA measurements with optical methods are used to validate and calibrate the electrical measurements.

According to a particular embodiment, simultaneous measurements of e.g. a sample library (e.g. of SNP's) is performed, whereafter the data obtained using the two measurements are correlated, in order to obtain a SNP library based on electrical detection of signals. This allows subsequent reliable identification of SNPs based on electrical detection only, thereby obviating the need for a label.

According to yet another particular embodiment, the electrical and optical detection methods are used consecutively. According to one embodiment, the melting temperature of a sample nucleic acid/nucleic acid probe is first determined by the electrical sensor on a sample which has not been amplified or/and or which has not been labeled. Thereafter one or more amplication steps and/or labelling steps are performed. At this stage the melting temperature determination can be performed using solely the optical method or using a combination of electrical and optical method to obtain results from two independent measuring methods.

The invention describes devices and methods wherein the melting temperature of ds nucleic acids is determined and MCA is performed using an optical method and/or an electrical method. The determination of the melting temperature by these methods and devices has a number of advantages. A change in optical properties is generally only observed when the sample nucleic acid is completely dissociated from the probe nucleic acid and becomes physically separated from the area of optical measurement. However prior to this total dissociation, a ds nucleic acid will locally denature. Using an electrical sensor, such preliminary denaturation is immediately monitored. Similarly, upon complete denaturation of the ds nucleic acid, the change in electrical signal is immediately detected, without having to wait for the physical removal of the sample. Consequently melting curves are sharper and more accurate. For the same reason the temperature gradient which is applied to the electrical sensor can be steeper, thereby shortening the time required for performing a MCA.

Other arrangements of the systems and methods embodying the invention will be obvious for those skilled in the art. It is to be understood that although particular embodiments, specific constructions and configurations, as well as materials, are discussed herein for devices and methods according to the present invention, various changes or modifications in form and detail may be made without departing from the scope and spirit of this invention.

### EXAMPLE 1

### Melting Temperature Determination by Fluorescent Monitoring

An oligonucleotide probe comprising for a hemochromatosis SNP (such as described in Star et al. (cited above, Table 2) is spotted on a carbon nanotube sensor. After drying, the nanosensor is placed in a reaction chamber and a buffer with a sample ss nucleic acid, previously labeled with a fluorescent label, suspected to comprise a mismatch is added. The reaction chamber is placed in a device equipped with electrodes to measure the conductance over the carbon nanotube sensor and equipped with optics to detect fluorescence at the surface of the carbon nanotube sensor. The reaction chamber is heated to 100° C. and gradually cooled down to anneal probe and labeled sample nucleic acid. The reaction chamber is then gradually heated at a rate of 0.1° C. per second. During the ramp (each 5 seconds) the electrical conductance of the carbon nanotube is detected and absorption of the sample nucleic acid/nucleic acid probe present on the surface of the nanotube sensor is measured optically. The raw data are first converted by taking the negative first derivative of both the fluorescence and conductance values. Final melting curves are thus reported as the three point-smoothed negative first derivative of fluorescence and conductance with respect to temperature versus temperature, optionally with a base line subtraction. The baseline correction for each data point is calculated by subtracting the slope from a linear regression line encompassing four data points immediately preceding and succeeding the current point.

1. A biosensor device (100) comprising:
a microchamber (101),
an electrical detection means (102) comprising an electric sensor placed within the microchamber, wherein said sensor is capable of detecting a change in an electric property of a double stranded nucleic acid present on its surface, and
a programmable heating element (103) capable of heating the microchamber.

2. The device according to claim 1, wherein the electric sensor comprises a nanostructure.

3. The device of claim 2 wherein the nanostructure is a carbon nanotube.

4. The device according to claim 1, characterized in that at least one single stranded nucleic acid is present on the electric sensor.

5. The device of claim 1, characterized in that the microchamber comprises at least one semi-transparent or transparent portion.

6. The device of claim 5, comprising an optical detection means capable of detecting a signal generated in the microchamber through the at least one semi-transparent or transparent portion of the microchamber.

7. The device according to claim 4 wherein the optical detection means is a fluorescence detector.

8. The device of claim 1, wherein the at least one electric sensors are arrayed.

9. The device of claim 1, further comprising a first substrate supporting the electric sensor.

10. A method for performing Melting Curve Analysis, the method comprising:
providing a single stranded nucleic acid probe on the surface of electric sensor in a microchamber,
contacting a sample comprising a single stranded nucleic acid target with the electric sensor in the microchamber so as to allow hybridization between the single stranded nucleic acid probe and the single strand nucleic acid target to a double stranded nucleic acid,
gradually heating the microchamber
detecting the melting temperature of the double stranded nucleic acid based on a changing electrical signal on the electric sensor.

11. The method according to claim 9, which further comprises the steps of:
detecting the melting temperature of the double stranded nucleic acid based on a changing optical signal; and
comparing the value obtained in step (d) with the value obtained in step (e).

12. A method for determining the presence of one or more nucleotide polymorphisms in a nucleic acid fragment of a gene in a sample using an electrical detection method, the method comprising:
determining the melting temperature of a library of nucleotide polymorphisms of the gene using an electrical detection means
simultaneously determining the melting temperature of nucleotide polymorphisms of the gene using optical detection means
correlating the values of obtained in the electrical detection means in step (a) with those obtained with the optical detection means of step (b) so as to obtain a library of values of melting points based on electrical detection
determining the melting point for the nucleic acid fragment of the sample using an electrical detection method
comparing the value obtained in step (d) with the library obtained in step (c) so as to obtain a reliable indication of the presence of the one or more polymorphisms.

13. A method for calibrating the device of claim 1 comprising the steps of:
determining the melting temperature of a double stranded nucleic acid using an electrical measurement method.
verifying the melting temperature of said double stranded nucleic acid obtained by said electrical measurement value one or more times by an optical method.
defining electrical measurement values corresponding to the melting temperature of said double stranded nucleic acid.

14. A method for measuring the hybridization between a sample nucleic acid and a nucleic acid probe comprising the steps of:
providing an electric sensor with the single stranded nucleic acid probe
applying a sample with sample nucleic acid under conditions wherein said sample nucleic acid can hybridize with said nucleic acid probe
gradually heating the hybridized nucleic acid on the electric sensor in a controlled way.
determining the melting temperature of the hybridized nucleic acid by way of the electrical sensor.

15. The method according to claim 1, further comprising the step of detecting, during the heating of the hybridized nucleic acid, the melting point of the hybridized nucleic acid by way of optical detection.

16. The method according to claim 15, wherein the sample nucleic acid is labeled with an optical label.

17. The method of claim 15, wherein the heating is performed at a speed of at least 1° C. per second.

18. The method of claim 15 wherein the detection is performed exclusively by electrical measurement using the electrical measurement values.

19. Use of the device of claim 1 for determining mismatches in a ds nucleic acid.

20. A reaction chamber of a nanosensing device comprising:
at least one electric sensor,
a substrate supporting the electric sensor, wherein said substrate is heat conducting, wherein said microchamber comprises at least one (semi)transparent portion.

21. The reaction chamber of claim 20, further comprising a programmable heating element capable of heating the microchamber.
